(19) United States
(12) Patent Application Publication
(10) Pub. No.: US 2004/0009202 A1
(43) Pub. Date: Jan. 15, 2004

(54) Skincare substance patch

(75) Inventor: Karl-Heinz Woller, Hamburg (DE)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 10/435,991

(22) Filed: May 12, 2003

Related U.S. Application Data
(63) Continuation of application No. PCT/EP01/12606, filed on Oct. 31, 2001.

(30) Foreign Application Priority Data
Nov. 11, 2000 (DE) DE 100 56 010.5,

(51) Int. Cl. A61K 7/00

(52) U.S. Cl. 424/401

(57) ABSTRACT

A matrix patch for the controlled delivery of cosmetic skincare additives to the skin, comprising a flexible outer layer and a water-insoluble pressure-sensitive adhesive matrix, the pressure sensitive matrix being free from mineral oils and tackifier resins and comprising:

a) synthetic framework polymers based on polyisobutylene, at from 25 to 90% by weight

b) amorphous poly-α-olefin at from 5 to 30% by weight,

c) an insoluble, especially hydrophilic, filler at from 0 to 60% by weight, and

d) cosmetic skincare additives at from 0.2 to 30% by weight.

### CROSS-REFERENCE TO RELATED APPLICATIONS

This is a continuation application of PCT/EP01/12606, filed Oct. 31, 2001, which is incorporated herein by reference in its entirety, and also claims the benefit of German Priority Application No. 100 56 010.5, filed Nov. 11, 2000.

### FIELD OF THE INVENTION

The invention relates to patches with skincare substances in the adhesive matrix.

### BACKGROUND OF THE INVENTION

The mechanism of action of patches for administering cosmetic care substances into the skin is subject to a functional principle analogous to that of transdermal therapeutic systems.

Transdermal therapeutic systems (TTS) represent patch-type, in particular drug-doped systems. The time-dependent release of the substance, for example the drug, from the TTS occurs as a function of its TTS/skin distribution coefficient and of its diffusion in the region of the TTS and the skin. Both factors can be influenced by the composition of the matrix in which the substance is incorporated, allowing a direct influence to be exerted on the amount released per unit time and the duration of activity. In the ideal case a first-order release kinetics is obtained, allowing equal quantities to be released per unit time. These properties of the TTS prevent repeated application and exposure of the skin to high concentrations and hence prevent the irritation to the skin that is inevitable on repeated application of liquid and semisolid substance forms. Should side effects nevertheless occur in the course of a TTS application, further exposure can be halted immediately by removing the TTS. Lining the substance matrix with a film which prevents emergence of the substance on the side of the TTS remote from the body boosts the transdermal effect.

A final advantage of the TTS is the greatly improved user compliance, attributable to the quick and easy application and the long activity of the TTS.

Marked disadvantages which must be resolved when developing the TTS include the release of the substance from the system and its sufficient uptake into the skin in order to achieve the cosmetically effective concentration. In order to be able to control these parameters use is made of hydrocolloids, solubilizers, and enhancers, which allow improved solubility and diffusion and also a more rapid passage of the substance from the TTS into the skin.

An embodiment of such transdermal systems which has been thoroughly described in the technical literature is that of matrix systems or monolithic systems, in which the substance is incorporated directly into the pressure sensitive adhesive. In the ready-to-apply product, a pressure sensitive substance matrix of this kind is equipped on one side with a backing, which is impervious to the substance, while on the other side there is a backing film which is equipped with a release layer and which is removed prior to application to the skin (Kleben&Dichten, No.42, 1998, pp. 26 to 30).

One basic requirement of a TTS is a very good adhesion to skin, which must be maintained over the entire period of the intended metering. One frequently observed side effect, however, is the occurrence of skin irritations, which occur in particular when a TTS is applied for a long time or repeatedly to the same region of the body. The principal cause of such irritation is the ingredients of the pressure sensitive matrix. Painful redetachment of the active substance patch after a prolonged period of wear is another frequent observation.

Repeated and long-lasting applications of pressure sensitive systems on the same regions of the human body are encountered in particular in the area of stoma care. Here, hydrocolloids have been used for a long time and with great success as the pressure sensitive adhesive. Hydrocolloids consist in principle of a hydrophobic pressure-sensitive adhesive polymer matrix based on synthetic rubbers, with insoluble hydrophilic fillers based on alginates, cellulose or pectins, for example, present in disperse distribution in this matrix. In the development of hydrocolloids for stoma care, however, the adhesion properties to moist skin and the ability to take up fluid stand in the foreground.

As early as 1967 U.S. Pat. No. 3,339,546 described a hydrocolloid based on polyisobutylenes for use in the buccal cavity. A great disadvantage of the early systems was the poor integrity of the matrices, i.e., the breakdown and disintegration of the pressure sensitive matrix when absorbing fairly large amounts of fluid.

Later developments therefore aimed at solving this problem, and a number of solutions are shown in the literature. U.S. Pat. No. 4,393,080 describes for example a hydrocolloid system based on elastomers, where high molecular mass hydrophilic fillers are used, which promote the consistency even of the swollen system. Further documents describe solutions involving the crosslinking of the elastomer matrix, either physically or chemically.

Physical crosslinking can be brought about, for example, by the use of phase-separating block polymers based on poly(styrene-b-isoprene-b-styrene) (SIS), poly(styrene-b-isoprene-b-styrene) (SBS) or poly[styrene-b-(ethylene-stat.-butylene)-b-styrene] (SEBS). One of the first such systems is described for example in DE 28 22 535.

Chemical crosslinking can be brought about for example by electron beam or γ-irradiation of the hydrocolloid matrix. A prerequisite here is that sufficiently reactive structural elements are present in the pressure sensitive matrix. This can be done by way of example, as described in U.S. Pat. No. 4,477,325, by compounding with an ethylene-vinyl acetate copolymer.

Although the aforementioned inventions describe and solve the technical problem of the cohesiveness of swollen hydrocolloids, they do not address the problem of skin irritation as a result of repeated application.

A document which does concern itself with possible occurrences of skin irritation is WO 98/01167 A1. There, aloe vera extract is used in order to prevent inflammatory skin changes and also infections in the context of stoma care. The system described, however, consists merely of a low molecular polyisobutylene polymer framework, and so the above-described problem of the cohesiveness of the hydrocolloid matrix continues to exist. Additionally, the composition described uses tackifier resins, the allergenic potential of which is known. Information on the suitability of such a system for the controlled delivery of skin-friendly cosmetic substances are contained in neither this nor other of the cited texts.

Transdermal therapeutic systems are generally applied to healthy, intact skin. Here specifically it is particularly important not to irritate, let alone damage, the intact skin. Sufficient cohesiveness is a further necessity in order to allow the substance patch to be removed without residue after the end of the period of wear.

Polyisobutylenes have long been used as a framework substance in the compounding of pressure sensitive adhesives. As compared with other known elastomers, synthetic polymers based on isobutylene offer a series of advantages. As a result of their synthetic preparation they are free from unwanted ingredients; as a result of their complete saturation they are highly stable to oxidation; and depending on their molecular weight they feature an inherent tack which can be adjusted.

For application to skin in particular, therefore, they are given preference over other elastomers. For example, the allergenic potential of natural rubber, owing to its natural impurities, is well known. Other synthetic rubbers based on styrene and isoprene and/or butadiene are highly sensitive to oxidation, necessitating the addition of further additives. Their hydrogenated derivatives based on poly[styrene-b-(ethylene-stat-propylene)-b-styrene] (SE PS) or poly[styrene-b-(ethylene-stat.-butylene)-b-styrene] (SEBS) are, it is true, more stable to oxidation, but lack inherent tack. For this reason their use as pressure sensitive adhesives unavoidably necessitates compounding with tackifier resins, as described in EP 0 651 635 B1, for example. These resins are compositions of matter which are normally very poorly defined, and frequently are based on rosin. As a result, it is impossible to rule out an allergenic potential here as well.

The use of polyisobutylenes for transdermal therapeutic systems is described in DE 33 47 278 A1 and DE 33 47 277 A1. There, however, they are used in combination with either olefinic diene rubbers or tackifier resins, which again have the disadvantages described above. Also described is the addition of amorphous poly-α-olefins, although their effect on the system as a whole is not elucidated. The use of fillers is not mentioned in this description.

The use of PIB for transdermal systems without the addition of tackifier resins is described in U.S. Pat. No. 4,559,222. In that case, however, it is necessary to use very large amounts of mineral oil, the ratio of mineral oil to PIB in accordance with that invention being at least 1. Moreover, the system is restricted to active substances which are of moderate solubility in mineral oil. This also exerts a plasticizing effect on the matrix. At least 6% by weight of colloidal silica is used as a filler.

WO 96/22083 A1 describes a system for the transdermal administration of nicotine, which is based on polyisobutylene but without the addition of mineral oil. Here, however, the necessary tack of the adhesive is achieved through the use of tackifier resins. In terms of skin compatibility these possess the abovementioned drawbacks. A plasticizing effect, which further positively influences the adhesive properties of the matrix, is achieved through the active substance which is soluble in the PIB matrix. This principle of compounding, however, very greatly restricts the selection of the active substances which can be administered by way of this matrix.

U.S. Pat. No. 5,508,038 addresses the problem of skin irritation through the possible use of tackifier resins, the only one of the said connections to do so.

The use of amorphous poly-α-olefins in pressure sensitive adhesives in general is known in the literature. U.S. Pat. No. 4,186,258 was one of the first documents to specify the use of this class of substance in the field of pressure sensitive hotmelt adhesives. A specific field of use is not mentioned in that document.

U.S. Pat. No. 5,262,216 describes the use of these materials together with tackifier resins for hotmelt pressure sensitive adhesives specifically for use in self-adhesive labels. This class of polymer is praised in particular there for its outstanding UV stability and aging stability. The human field of use is not mentioned.

WO 98/54268 A1, on the other hand, specifically describes the use of amorphous poly-α-olefins for applications on human skin. Here, additionally, amorphous poly-α-olefins are used in combination with fillers. Described specifically, however, is the field of use of wound coverage. In this field of use, a feature of the amorphous poly-a-olefins is their outstanding radiation resistance, allowing the production in accordance with that invention of products which can be effectively sterilized for wound care. Additionally, amorphous poly-α-olefins are used here in combination with tackifier resins. The aspect of reduced skin irritation is not mentioned overall.

The systems described to date for transdermal administration of a substance do not include organic fillers. However, these fillers in particular are responsible for the skinfriendliness of the aforedescribed pressure sensitive adhesives for stoma care. By means of these fillers it is possible for the moisture given off by the skin during the period for which the patch is worn to be absorbed very effectively. The resultant climate below the patch leads to a marked reduction in the occurrence of skin maceration.

An invention of a patch using water-swellable fillers is described by EP 0 186 019 A1. There, however, the positive influence of the organic filler on the rate of release of the active substance is described. The filler content of that invention is limited to 30% by weight. The aspect of the reduction of skin irritation is not addressed. Additionally, the systems described are realized with the use of tackifier resins.

It is an object of the present invention to develop a matrix for the controlled delivery of a skin-friendly substance, said matrix not only possessing excellent cohesiveness but also being composed of particularly skin-friendly components or being able to be realized with the complete omission of skin irritant components such as tackifier resins, for example. Planned additionally is a production process which manages with the complete omission of solvent; furthermore, the stated side effects of the pressure sensitive adhesive for transdermal systems—skin irritation and painful redetachment—are to be avoided, giving a substantial increase in wear comfort for the patient. The object is therefore to provide a matrix system based on polyisobutylene which can be produced without conventional tackifier resins and mineral oil in a solvent-free production process.

### SUMMARY OF THE INVENTION

This object is achieved by means of a matrix patch in accordance with the main claim. The subclaims relate to advantageous embodiments of the patch of the invention. The invention further embraces processes for producing such patches.

The invention accordingly provides a matrix patch for the controlled delivery of cosmetic skincare additives to the skin, comprising a flexible outer layer and a water-insoluble pressure-sensitive adhesive matrix which contains additives, the pressure sensitive matrix being free from mineral oils and tackifier resins and being composed of

a) synthetic framework polymers based on polyisobutylene, at from 25 to 90% by weight,

b) amorphous poly-a-olefin at from 5 to 30% by weight,

c) an insoluble, especially hydrophilic, filler at from 0 to 60% by weight, and

d) cosmetic skincare additives at from 0.2 to 30% by weight.

In an advantageous embodiment of the matrix patch the polyisobutylene is composed of high molecular weight PIB at from 5 to 55% by weight and low molecular weight PIB at from 20 to 60% by weight.

A typical pressure sensitive adhesive of the invention is therefore composed of the following:

| | | | | |
|---|---|---|---|---|
| high molecular | 5-55% | by weight | preferably 25-45% | by wt. |
| wt. PIB | | | | |
| low molecular | 20-60% | by weight | preferably 30-50% | by wt. |
| wt. PIB | | | | |
| amorphous | 5-30% | by weight | preferably 5-10% | by weight |
| poly-α-olefin | | | | |
| hydrophilic | 0-60% | by weight | preferably 0-30% | by weight |
| filler | | | | |
| cosmetic skin- | 0.2-10% | by weight | preferably 0.5-5% | by wt. |
| care additives | | | | |

As an option it is also possible to add up to 20% by weight of a permeation promoter (lipophilic solubilizer/enhancer) such as decyl oleate, isopropyl myristate or isopropyl palmitate (IPM or IPP, respectively).

### DETAILED DESCRIPTION OF THE PREFERRED EMBODIMENTS

The specified formula constituents are defined in more detail as follows:

High molecular weight PIB: Polyisobutylene having a weight-average molecular weight (M_{w}) of from 300,000 to 1,100,000, preferably between 650,000 and 850,000. Such polymers are available commercially for example under the trade names Oppanol B100 (BASF) or Vistanex MM-L80 (Exxon).

Low molecular weight PIB: Polyisobutylene having a weight-average molecular weight (M_{w}) of from 40,000 to 300,000, preferably between 60,000 and 100,000. Such polymers are available commercially for example under the trade names Oppanol B15 (BASF) or Vistanex LMMH (Exxon).

Amorphous poly-α-olefin: Amorphous copolymers based on ethylene and propylene, butylene or 1-hexene. The preferred weight-average molecular weight (Mw) is from 5,000 to 100,000, more preferably between 10,000 and 30,000. Such polymers are available commercially for example under the trade names Eastoflex® (Eastman) or Vestoplast® (Hüls).

Particularly hydrophilic filler: Hydrophilic particles insoluble in the stated polymer matrix and based on cellulose. Preference is given to an average particle size of less than or equal to 100 μm with a surface which is as uniform as possible. Such materials are available commercially for example under the trade names Avicel (FMC) and Elcema (Degussa-Hüls).

In accordance with the invention, the cosmetic skincare additives (one or more compounds) can be selected very advantageously from the group consisting of lipophilic additives, and in particular from the following group: acetylsalicylic acid, atropine, azulene, hydrocortisone and its derivatives, e.g. hydrocortisone 17-valerate, vitamins, e.g. ascorbic acid and its derivatives, vitamins of the B and D series, very advantageously vitamin B₁, vitamin B₁₂, vitamin D₁, and also bisabolol, unsaturated fatty acids, namely the essential fatty acids (often also called vitamin F), especially gamma-linolenic acid, oleic acid, eicosapentaenoic acid, docosahexaenoic acid, and derivatives thereof, chloramphenicol, caffeine, prostaglandins, thymol, camphor, extracts or other products of plant and animal origin, e.g., evening primrose oil, borage oil or currant seed oil, fish oils, cod-liver oil or else ceramides and ceramidelike compounds and the like.

It is also advantageous to select the additives from the group consisting of fat-restoring substances, examples being purcellin oil, Eucerito, and Neocerit®.

With particular advantage the additive or additives is or are selected further from the group of the NO synthase inhibitors, especially when the preparations of the invention are to be used for the treatment and prophylaxis of the symptoms of intrinsic and/or extrinsic skin aging and also for the treatment and prophylaxis of the harmful effects of ultraviolet radiation on the skin. A preferred NO synthase inhibitor is nitroarginine.

The additive(s) is/are further advantageously selected from the group which includes catechins and bile esters of catechins and aqueous or organic extracts from plants or parts of plants which contain catechins or bile esters of catechins, such as, for example, the leaves of the Theaceae plant family, in particular of the species Camellia sinensis (green tea). Particularly advantageous are typical ingredients thereof (such as polyphenols or catechins, caffeine, vitamins, sugars, minerals, amino acids, and lipids, for example).

Catechins are a group of compounds which can be regarded as hydrogenated flavones or anthocyanidines and are derivatives of “catechin” (catechol, 3,3′,4′,5,7-flavanpentaol, 2-(3,4-dihydroxyphenyl)chroman-3,5,7-triol). Epicatechin ((2R,3R)-3,3′,4′,5,7-flavanpentaol) is also an advantageous additive for the purposes of the present invention.

Also advantageous are plant extracts combining catechins, in particular extracts of green tea, such as extracts from leaves of plants of the species Camellia spec., very particularly the tea varieties Camellia sinenis, C. assamica, C. taliensis, and C. irrawadiensis and hybrids of these with, for example, Camellia japonica.

Preferred additives also include polyphenols or catechins from the group (−)-catechin, (+)-catechin, (−)-catechin gallate, (−)-gallocatechin gallate, (+)-epicatechin, (−)-epicatechin, (−)-epicatechin gallate, (−)-epigallocatechin, and (−)-epigallocatechin gallate.

Flavone and its derivatives (also often collectively called “flavones”) are also advantageous additives for the purposes of the present invention. They are characterized by the following parent structure (substitution positions indicated):

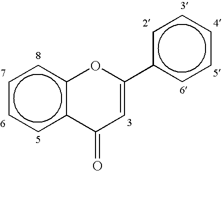

Some of the more important flavones which can also preferentially be used in preparations according to the invention are listed in the table below:

OH substitution positions

| | 3 | 5 | 7 | 8 | 2′ | 3′ | 4′ | 5′ |
|---|---|---|---|---|---|---|---|---|
| Flavone | − | − | − | − | − | − | − | − |
| Flavonol | + | − | − | − | − | − | − | − |
| Chrysin | − | + | + | − | − | − | − | − |
| Galangin | + | + | − | − | − | − | − | − |
| Apigenin | − | + | + | − | − | − | + | − |
| Fisetin | + | − | + | − | − | + | + | − |
| Luteolin | − | − | + | − | − | + | + | − |
| Kampferol | + | + | + | − | − | − | + | − |
| Quercetin | + | + | + | − | − | + | + | − |
| Morin | − | + | + | − | + | − | + | − |
| Robinetin | + | − | + | − | − | + | + | + |
| Gossypetin | + | + | + | + | − | + | + | − |
| Myricetin | + | + | + | − | − | + | + | + |

In nature, flavones are usually in glycosylated form.

According to the invention, the flavonoids are preferably chosen from the group of substances of the generic structural formula

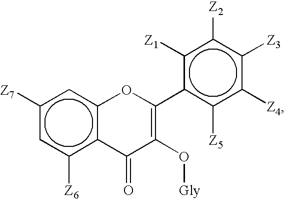

where Z₁ to Z₇, independently of one another, are chosen from the group consisting of H, OH, alkoxy and hydroxyalkoxy, where the alkoxy and hydroxyalkoxy groups can be branched or unbranched and have 1 to 18 carbon atoms, and where Gly is chosen from the group of mono- and oligoglycoside radicals.

According to the invention, the flavonoids can, however, also advantageously be chosen from the group of substances of the generic structural formula

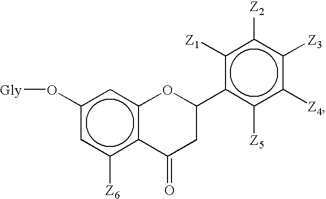

where Z₁ to Z₆, independently of one another, are chosen from the group consisting of H, OH, alkoxy and hydroxyalkoxy, where the alkoxy and hydroxyalkoxy groups can be branched or unbranched and have 1 to 18 carbon atoms, and where Gly is chosen from the group of mono and oligoglycoside radicals.

Preferably, such structures can be chosen from the group of substances of the generic structural formula

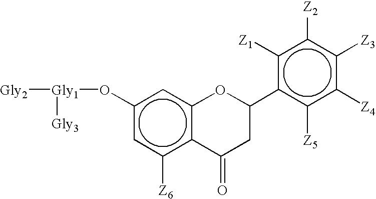

where Gly₁, Gly₂ and Gly₃, independently of one another, are monoglycoside radicals. Gly₂ and Gly₃ can also, individually or together, represent saturations by hydrogen atoms.

Preferably, Gly₁, Gly₂ and Gly₃, independently of one another, are chosen from the group of hexosyl radicals, in particular of rhamnosyl radicals and glucosyl radicals. However, other hexosyl radicals, for example allosyl, altrosyl, galactosyl, gulosyl, idosyl, mannosyl and talosyl, can also be used advantageously in some circumstances. It may also be advantageous according to the invention to use pentosyl radicals.

Z₁ to Z₅ are, independently of one another, advantageously chosen from the group consisting of H, OH, methoxy, ethoxy and 2-hydroxyethoxy, and the flavone glycosides have the structure

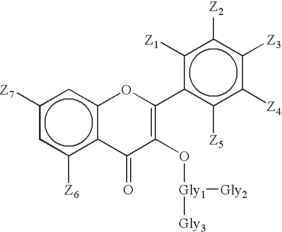

The flavone glycosides according to the invention are particularly advantageously chosen from the group shown by the following structure:

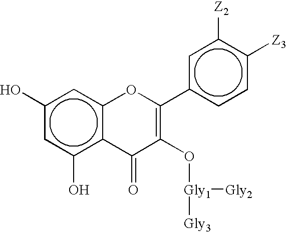

where Gly₁, Gly₂ and Gly₃, independently of one another, are monoglycoside radicals. Gly₂ and Gly₃ can also, individually or together, represent saturations by hydrogen atoms.

Preferably, Gly₁, Gly₂ and Gly₃, independently of one another, are chosen from the group of hexosyl radicals, in particular of rhamnosyl radicals and glucosyl radicals. However, other hexosyl radicals, for example allosyl, altrosyl, galactosyl, gulosyl, idosyl, mannosyl and talosyl, can also advantageously be used in some circumstances. It may also be advantageous according to the invention to use pentosyl radicals.

For the purposes of the present invention, it is particularly advantageous to choose the flavone glucoside(s) from the group consisting of α-glucosylrutin, α-glucosylmyricetin, α-glucosylisoquercitrin, α-glucosylisoquercetin and α-glucosylquercitrin. Particular preference is given, according to the invention, to α-glucosylrutin.

Also advantageous according to the invention are naringin (aurantin, naringenin-7-rhamno-glucoside), hesperidin (3′,5,7-trihydroxy-4′-methoxyflavanone-7-rutinoside, hesperidoside, hesperetin-7-O-rutinoside), rutin (3,3′,4′,5,7-pentahydroxyflavone-3-rutinoside, quercetin-3-rutinoside, sophorin, birutan, rutabion, taurutin, phytomelin, melin), troxerutin (3,5-dihydroxy-3′,4′,7-tris(2-hydroxyethoxy)flavone-3-(6-0-(6-deoxy-α-L-mannopyranosyl)-β-D-glucopyranoside)), monoxerutin (3,3′,4′, 5-tetrahydroxy-7-(2-hydroxyethoxy)flavone-3-(6-O-(6-deoxy-α-L-mannopyranosyl)-β-D-glucopyranoside)), dihydrorobinetin (3,3′,4′,5′,7-pentahydroxyflavanone), taxifolin (3,3′,4′,5,7-pentahydroxy-flavanone), eriodictyol-7-glucoside (3′,4′,5,7-tetrahydroxyfiavanone-7-glucoside), flavanomarein (3′,4′,7,8-tetrahydroxyflavanone-7 glucoside) and isoquercetin (3,3′,4′,5,7-pentahydroxyflavanone-3-(β-D-glucopyranoside).

It is also advantageous to choose the additive(s) from the group of ubiquinones and plastoquinones.

Ubiquinones are distinguished by the structural formula

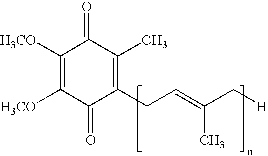

and are the most widespread and thus the most investigated bioquinones. Ubiquinones are referred to depending on the number of isoprene units linked in the side chain as Q-1, Q-2, Q-3 etc., or depending on the number of carbon atoms, as U-5, U-10, U-15 etc. They preferably appear with defined chain lengths, e.g., in some microorganisms and yeasts where n=6. In most mammals, including humans, Q10 predominates. Coenzyme Q10 is particularly advantageous and is characterized by the following structural formula:

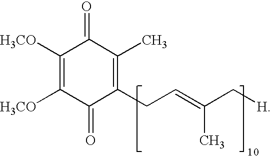

Plastoquinones have the general structural formula

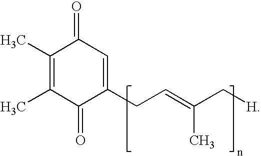

Plastoquinones differ in the number n of isoprene radicals and are referred to accordingly, e.g., PQ-9 (n=9). In addition, other plastoquinones with varying substituents on the quinone ring exist.

Creatine and/or creatine derivatives are preferred additives for the purposes of the present invention. Creatine is characterized by the following structure:

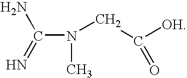

Preferred derivatives are creatine phosphate and creatine sulfate, creatine acetate, creatine ascorbate, and the derivatives esterified at the carboxyl group with mono- or polyfunctional alcohols.

A further advantageous additive is L-carnitine [3-hydroxy-4-(trimethylammonio)-butyrobetaine]. Acylcarnitines chosen from the group of substances of the following general structural formula

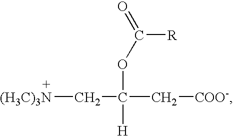

where R is chosen from the group of branched and unbranched alkyl radicals having up to 10 carbon atoms, are also advantageous additives for the purposes of the present invention. Preference is given to propionylcarnitine and, in particular, acetylcarnitine. Both enantiomers (D and L form) can be used advantageously for the purposes of the present invention. It may also be advantageous to use any enantiomer mixtures, for example a racemate of D and L form.

Further advantageous additives are sericoside, pyridoxol, vitamin K, biotin, and aroma substances.

The list of said additives and additive combinations which can be used in the preparations according to the invention is, of course, not intended to be limiting. The additives can be used individually or in any combinations with one another.

Pharmaceutically active substances can then be added to the matrix of the active substance matrix patch, in particular at from 0.1 to 25% by weight, very particularly at from 0.5 to 10% by weight.

Typical active substances, without making any claim to completeness, in the context of the present invention, include the following:

| Indication: | Active substance |
|---|---|
| Antimycotics | Nafitine |
| | Amorrolfine |
| | Tolnaftate |
| | Ciclopirox |
| Antiseptics | Thymol |
| | Eugenol |
| | Triclosan |
| | Hexachlorophene |
| | Benzalkonium chloride |
| | Clioquinol |
| | Quinolinol |
| | Undecenoic acid |
| | Ethacridine |
| | Chlorhexidine |
| | Hexetidine |
| | Dodicine |
| | Iodine |
| Nonsteroidal antirheumatics | Glycol salicylate |
| | Flufenamic acid |
| | Ibuprofen |
| | Etofenamate |
| | Ketoprofen |
| | Piroxicam |
| | Indomethacin |
| Antipruritics | Polidocanol |
| | Isoprenaline |
| | Crotamiton |
| Local anesthetics | Benzocaine |
| Antipsoriatics | Ammonium bitumasulfonate |
| Keratolytics | Urea |
| | Salicylic acid |

In addition it is also possible to mention hyperemic substances such as natural active substances of cayenne pepper or synthetic active substances such as nonivamide, nicotinic acid derivatives, preferably benzyl nicotinate or propyl nicotinate, and antiphlogistics and/or analgesics.

The matrix is preferably produced in a process in which all of the components of the pressure sensitive matrix are homogenized in the melt without the addition of solvent. With particular preference, all of the components are processed in a continuous or batchwise operation at a temperature below 100° C.

The matrix features outstanding properties of adhesion to the skin, easy and painless redetachability, and, in particular, an extremely low potential to induce skin irritation. The production operation proceeds with the complete omission of solvents. If desired, the open adhesive side, that to be applied to the skin, can be lined with a redetachable protective covering layer.

In the context of the selection of the fillers it has surprisingly been found that fillers based on silica or on cellulose are especially suitable, the latter possessing an isotropic morphology and showing no tendency to swell on contact with water. Particularly suitable in this context are fillers having a particle size of less than or equal to 100 μm.

The use of hydrophilic fillers in an apolar matrix is known in the literature. Explicitly for use in transdermal therapeutic systems they are described in EP 0 186 019 A1. There, however, they are only in a concentration of up to 3 to 30% by weight, and no details of these fillers are mentioned. Experience shows that systems with a filler content of more than 30% by weight exhibit a marked loss in tack and become hard and brittle. As a result they lose the fundamental requirement of a transdermal therapeutic system. In the context of the present invention, however, it has been possible to show that fillers based on microcrystalline or amorphous cellulose can be employed at substantially higher concentrations, without adversely affecting the adhesive properties, when they possess an isotropic morphology with a particle size of not more than 100 μm. Higher filler contents are desirable in order to improve the wear properties particularly in the case of long-lasting and repeated application.

The hydrophilic fillers known from the materials for stoma care are integrated into the matrix of the invention, which serves to promote the skin compatibility.

The objective—the topical application of cosmetic additives, assisted where appropriate with the use of highly skin-compatible additives—can be promoted in the context of the present invention by adding permeation promoters such as fatty acid esters, for example.

Surprisingly it has been possible to realize the stated requirements in particular by means of a system which in addition to polyisobutylenes comprises amorphous poly-α-olefins in combination with amorphous or microcrystalline cellulose. This simple system contains as its polymer base exclusively synthetic ingredients whose quality can be monitored very effectively. As a result it is possible to a large extent to rule out allergenic reactions. The complete omission of poorly defined ingredients such as natural rubber or tackifier resins, for example, leads as a result to particularly skin-friendly matrices. Moreover, the adhesive properties of the formulation of the invention are very effectively adjustable. It is also possible to do without the further addition of additives to the system for the purpose of stabilization.

As already indicated, particularly skin-friendly systems can be realized on the basis of polyisobutylenes with the use of amorphous poly-α-olefins and also particulate cellulose filler.

The particular advantage of this raw material base lies in the use of exclusively fully saturated synthetic elastomers. These are very well defined and characterized; as a result, it is possible to rule out contamination with concomitant allergenic substances. As a result of the high degree of saturation these polymers which are used are very stable to oxidation. There is therefore no need for further addition of antioxidants and other stabilizers. The addition of such additives, such as is necessary when using natural rubber or unsaturated synthetic rubbers, always harbors the risk of skin incompatibility, owing to the chemical structure of the customary additives. Moreover, it represents an additional cost factor. In addition, all of the elastomers used possess an inherent tack, depending on the height of the molecular weight. As a result it is also possible to do without tackifier resins. Tackifier resins are compositions of matter which are frequently prepared on the basis of rosin and are very poorly defined. A uniform structural formula can be indicated only in the rarest of cases. This makes it more difficult to use tackifier resins as a raw material in drugs which are subject to approval, as in the present case of the transdermal therapeutic systems. Owing to the molecular-weight-dependent adhesion capacity to skin both of the polyisobutylenes and of the amorphous poly-α-olefins it is possible to adjust the adhesive properties of the system of the invention within a very wide range without having to change the chemistry of the base components. In many cases it is sufficient to vary the percentage proportions of the base components slightly in order to obtain desired product properties. The careful selection of unobjectionable raw materials of high skin compatibility leads to high expenditure in terms of both cost and time. It is therefore desirable to be able to set properties of a product by varying the percentage composition of the known raw materials. This avoids the time-consuming replacement of a complete raw material.

### EXAMPLES

### Examples 1 to 17

In order to examine the effect of different ingredients of the pressure sensitive matrix in terms of the skin adhesion, 17 comparison formulas were prepared in the context of a statistical experimental plan.

The adhesion of the adhesive systems to skin was tested in a wearing test by 6 volunteers. For this reason, the incorporation of the drug was omitted to start with. The specimens were evaluated within a school grade system on a scale from 1 to 6, with 1 denoting the best evaluation and 6 the worst evaluation.

The laboratory specimens were produced by the following general procedure:

A laboratory compounder equipped with duplex blades was charged at a temperature of 100° C. with the stated amount of Vistanex MM L80 and this initial charge was kneaded for one hour until the material is crumblike. Subsequently, in succession, the stated amounts of Vistanex LM MH, tackifier resin, and amorphous poly-a-olefin were added and kneading was carried out for a further hour until the material was homogeneous. Finally the filler was added in the stated amount and kneading was continued for an hour. After cooling, the material was removed from the compounder. The composition was subsequently pressed between siliconized paper using a hot press at about 120° C., to a thickness of 500 μm. These specimens were laminated on one side with a backing layer of polypropylene and on the side opposite this layer were lined with a siliconized polyester film.

Specimens measuring about 2.0×6.0 cm² were punched from this construction, and possess the form of standard commercial bandage strips.

The specimens thus produced were adhered by the volunteers to the inside of the lower arm and were worn for a period of 6 h. Evaluations were made of the initial tack of the specimens to skin and also the adhesion of the specimens over a period of 6 h.

TABLE 1
Exemplary formulas 1 to 17
Table 1 contains the exemplary formulas 1 to 17 processed by
this procedure.

| | PIB I | PIB II | Modifier | Filler |
|---|---|---|---|---|
| Example 1 | 17.0 | 30.0 | 5.0/I | 48.0/I |
| Example 2 | 20.0 | 50.0 | 5.0/II | 25.0/I |
| Example 3 | 10.0 | 30.0 | 20.0/III | 40.0/I |
| Example 4 | 10.0 | 50.0 | 20.0/III | 20.0/I |
| Example 5 | 17.0 | 43.0 | 20.0/III | 20.0/I |
| Example 6 | 7.0 | 38.0 | 5.0/IV | 50.0/I |
| Example 7 | 10.0 | 30.0 | 20.0/I | 40.0/II |
| Example 8 | 7.0 | 50.0 | 5.0/II | 38.0/II |
| Example 9 | 20.0 | 50.0 | 5.0/II | 25.0/II |
| Example | 7.0 | 38.0 | 5.0/III | 50.0/II |
| 10 | | | | |
| Example | 17.0 | 43.0 | 20.0/IV | 20.0/II |
| 11 | | | | |
| Example | 20.0 | 30.0 | 20.0/IV | 30.0/II |
| 12 | | | | |
| Example | 7.0 | 50.0 | 20.0/I | 23.0/III |
| 13 | | | | |
| Example | 17.0 | 30.0 | 20.0/II | 33.0/III |
| 14 | | | | |
| Example | 20.0 | 30.0 | 5.0/III | 45.0/III |
| 15 | | | | |
| Example | 10.0 | 50.0 | 5.0/IV | 35.0/III |
| 16 | | | | |
| Example | 14.0 | 39.0 | 12.0/IV | 35.0/III |
| 17 | | | | |

The following raw materials were used:

PIB I: Polyisobutylene, Vistanex MM L80, Exxon Chemical

PIB II: Polyisobutylene, Vistanex LM MH, Exxon Chemical

Modifying ingredients:

I: Aliphatic/aromatic hydrocarbon resin, Escorez 2101, Exxon Chemical

II: Hydrogenated polycyclopentadiene resin, Escorez 5300 I, Exxon Chemical

III: Hotmelt adhesive, Duro Tack H 1540, National Starch

IV: Amorphous poly-a-olefin, Eastoflex E 1003, Eastmann

Fillers:

I: Cellulose fibers, Just Fiber, International Filler of Belgium

II: Microcrystalline cellulose, Avicel PH 101, FMC

III: Colloidal silica, HiSil, PPG Industries

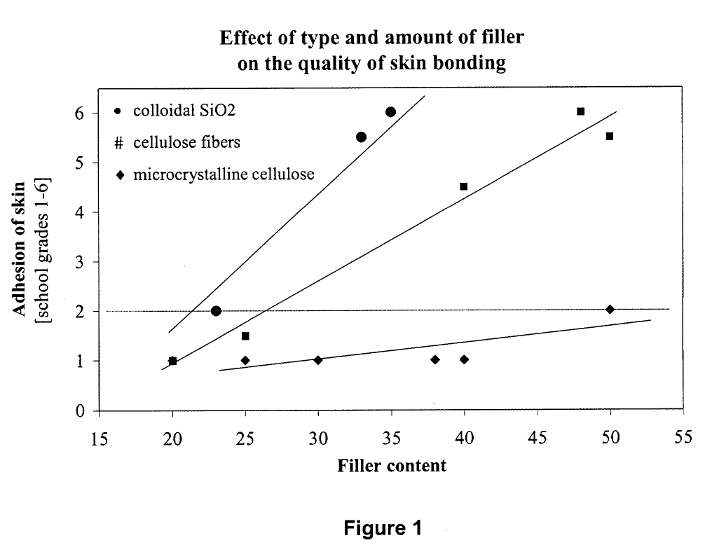

The adhesion properties to skin of the specimens thus produced were evaluated in accordance with a school grade system from 1 to 6. In this system 1 represents the best rating which can be awarded and 6 the poorest awardable rating. The results of this wear test are summarized in table 2 and also depicted in the form of a graph in FIG. 1.

TABLE 2
Evaluation of adhesion to skin

| Formula | 1 | 2 | 3 | 4 | 5 | 6 | 7 | 8 | 9 | 10 | 11 | 12 | 13 | 14 | 15 | 16 | 17 |
|---|---|---|---|---|---|---|---|---|---|---|---|---|---|---|---|---|---|
| Evaluation | 6 | 1.5 | 4.5 | 1 | 1 | 5.5 | 1 | 1 | 1 | 2 | 1 | 1 | 2 | 5.5 | ./. | 6 | 6 |

(./.: no measurable adhesion to skin)

From the figure it is clearly evident that the type and amount of the filler dominate the adhesive properties of the systems. Below an amount of about 20-25% by weight all of the systems, irrespective of the filler used, exhibit an adhesive behavior which is evaluated with at least “good” (2.0). This changes drastically when the amount of filler is raised to more than 30% by weight. Above a filler quantity of 30% by weight very good adhesive properties are exhibited by those systems in which, in accordance with the invention, cellulose with an average particle size of 50 pm is used.

### Examples 18 to 22

In order to examine the release from a system according to the invention laboratory specimens were prepared on the basis of the following formulas in accordance with the general preparation description.

| | |
|---|---|
| Example 18 | |
| Vistanex LM MH: | 48.33% by weight |
| Vistanex MM L80: | 28.00% by weight |
| Eastoflex PLS E1003D | 10.00% by weight |
| Cetiol V | 13.17% by weight |
| Bisabolol | 0.50% by weight |
| Example 19 | |
| Vistanex LM MH: | 48.33% by weight |
| Vistanex MM L80: | 33.00% by weight |
| Eastoflex PLS E1003D | 5.00% by weight |
| Cetiol V | 13.17% by weight |
| Vitamin E | 0.50% by weight |
| Example 20 | |
| Vistanex LM MH: | 48.33% by weight |
| Vistanex MM L80: | 33.00% by weight |
| Eastoflex PLS E1003D | 5.00% by weight |
| Cetiol V | 13.17% by weight |
| Vitamin F | 0.50% by weight |
| Example 21 | |
| Vistanex LM MH: | 43.33% by weight |
| Vistanex MM L80: | 38.00% by weight |
| Eastoflex PLS E1003D | 5.00% by weight |
| Cetiol V | 8.67% by weight |
| Camomile | 5.00% by weight |
| Example 22 | |
| Vistanex LM MH: | 43.33% by weight |
| Vistanex MM L80: | 38.00% by weight |
| Eastoflex PLS E1003D | 5.00% by weight |
| Cetiol V | 8.67% by weight |
| Jojoba | 5.00% by weight |

That which is claimed:

1. A matrix patch for the controlled delivery of cosmetic skincare additives to the skin, comprising a flexible outer layer and a water-insoluble pressure-sensitive adhesive matrix adjacent thereto, the pressure sensitive matrix being free from mineral oils and tackifier resins and comprising:
a) from 25 to 90% by weight of one or more polyisobutylene-based synthetic framework polymers,
b) from 5 to 30% by weight of one or more amorphous poly-a-olefins,
c) from 0 to 60% by weight of one or more insoluble fillers, and
d) from 0.2 to 30% by weight of one or more cosmetic skincare additives.

2. The matrix patch of claim 1, wherein the matrix comprises one or more poly-a-olefins in a concentration from 5 to 10% by weight.

3. The matrix patch of claim 1, wherein the polyisobutylene-based synthetic framework polymers include:
high molecular weight polyisobutylene (PIB) at from 5 to 55% by weight and low molecular weight PIB at from 20 to 60% by weight.

4. The matrix patch of claim 1, wherein the one or more insoluble fillers include hydrophilic fillers.

5. The matrix patch of claim 1, wherein the one or more fillers include fillers based on cellulose and its derivatives.

6. The matrix patch of claim 1, wherein the one or more fillers include hydrophilic fillers based on cellulose and its derivatives having an average particle size from 20 to 60 μm.

7. The matrix patch of claim 1, wherein the matrix contains includes cosmetic skincare additives selected from the group consisting of acetylsalicylic acid, atropine, azulene, hydrocortisone and its derivatives, vitamins, bisabolol, unsaturated fatty acids, chloramphenicol, caffeine, prostaglandins, thymol, camphor, extracts or other products of plant and animal origin, fish oils, and ceramides and ceramidelike compounds.

8. The matrix patch of claim 7, wherein the hydrocortisone and its derivatives is selected from the group consisting of hydrocortisone 17-valerate; the vitamins are selected from the group consisting of ascorbic acid and its derivatives and vitamins of the B and D series; the unsaturated fatty acids are selected from the group consisting of gamma-linolenic acid, oleic acid, eicosapentaenoic acid, docosahexaenoic acid, and derivatives thereof; the extracts or other products of plant and animal origin are selected from the group consisting of evening primrose oil, borage oil and currant seed oil; and the fish oil is cod-liver oil.

9. The matrix patch of claim 1, further comprising up to 30% by weight of one or more permeation promoters.

10. The matrix patch of claim 9, wherein the matrix comprises one or more permeation promoters in a concentration from 5 to 15% by weight.

11. The matrix patch of claim 1, wherein the matrix comprises cosmetic skincare additives in a concentration from 0.2 to 10% by weight.

12. The matrix patch of claim 1, wherein the matrix comprises cosmetic skincare additives in a concentration from 0.5 to 5% by weight.

13. The active substance matrix patch of claim 1, further comprising from 0.1 to 25% by weight of one or more pharmaceutically active substances.

14. The active substance matrix patch of claim 13, wherein the one or more pharmaceutically active substances are in a concentration from 0.5 to 10% by weight.

15. A process for producing a matrix patch of claim 1, wherein all the components of the pressure sensitive matrix are homogenized in a melt with the omission of solvent.

16. The process for producing a matrix patch of claim 15, characterized in that all the components are processed in a continuous or batchwise operation at a temperature below 100° C.

17. A process for making an active substance matrix patch for the controlled delivery of cosmetic skincare additives to the skin, comprising the steps of:
producing a homogenized melt comprising from 25 to 90% by weight of one or more polyisobutylene-based synthetic framework polymers, from 5 to 30% by weight of one or more amorphous poly-a-olefins, from 0 to 60% by weight of one or more insoluble fillers, and from 0.2 to 30% by weight of one or more cosmetic skincare additives, said melt being free of solvents, mineral oils and tackifier resins; and
applying the melt to a flexible backing layer.

18. The process for producing an active substance matrix patch of claim 17, wherein said producing step comprises producing a melt at a temperature below 100° C.
